(19) United States
(12) Patent Application Publication
(10) Pub. No.: US 2005/0250096 A1
(43) Pub. Date: Nov. 10, 2005

(54) Microorganism detection using bacteriophage amplification

(75) Inventors: John H. Wheeler, Boulder, CO (US); Jon Rees, Golden, CO (US); G. Scott Gaisford, Denver, CO (US)

Correspondence Address: PATTON BOGGS, 1660 LINCOLN ST, SUITE 2050, DENVER, CO, 80264, US

(73) Assignee: Microphage, Incorporated, Longmont, CO (US)

(21) Appl. No.: 11/147,800

(22) Filed: Jun. 7, 2005

Related U.S. Application Data
(63) Continuation-in-part of application No. 10/823,294, filed on Apr. 12, 2004.
(60) Provisional application No. 60/544,437, filed on Feb. 13, 2004.
(60) Provisional application No. 60/557,962, filed on Mar. 31, 2004.
(60) Provisional application No. 60/577,845, filed on Jun. 7, 2004.
(60) Provisional application No. 60/607,941, filed on Sep. 7, 2004.

(51) Int. Cl. C12Q 1/70; C12Q 1/04; C12Q 1/68

(52) U.S. Cl. 435/5; 435/34; 435/6

(57) ABSTRACT

A method of detecting the presence or absence of a target microorganism in a sample to be tested comprising: combining with the sample, bacteriophage capable of infecting the target microorganism to create a bacteriophage exposed sample; providing conditions to the bacteriophage exposed sample sufficient to allow the bacteriophage to infect the target microorganism to create injected bacteriophage nucleic acid, additional bacteriophage nucleic acid, intermediate bacteriophage protein, or additional bacteriophage protein; and assaying the nucleic acid or protein to determine the presence or absence of the target microorganism. The microorganism is preferably dissociated or lysed prior to the completion of the bacteriophage replication process. The sample is an insitu sample and the microorganism is isolated from the insitu matrix. The assaying comprises comparing the test nucleic acid or protein level to a reference level.

### CROSS REFERENCE TO RELATED APPLICATIONS

This application is a continuation-in-part of U.S. patent application Ser. No. 10/823,294 filed Apr. 12, 2004, which in turn claims the benefit of U.S. Provisional Application No. 60/544,437 filed Feb. 13, 2004 and U.S. Provisional Application No. 60/557,962 filed Mar. 31, 2004. Additionally, the present application claims the benefit of U.S. Provisional Application Ser. No. 60/577,845 filed Jun. 7, 2004 and U.S. Provisional Application Ser. No. 60/607,941 filed Sep. 7, 2004. All of the above patent applications, both provisional and non-provisional, are hereby incorporated by reference to the same extent as though fully contained herein.

### BACKGROUND OF THE INVENTION

1. Field of the Invention

The invention relates generally to the field of detection of microscopic living organisms, and more particularly to the detection of bacteria utilizing bacteriophage.

2. Statement of the Problem

Standard microbiological methods for detection of microorganisms have relied on substrate-based assays to test for the presence of specific bacterial pathogens. See Robert H. Bordner, John A. Winter, and Pasquale Scarpino, Microbiological Methods For Monitoring The Environment, EPA Report No. EPA-600/8-78-017, U.S. Environmental Protection Agency, Cincinnati, Ohio, 45268, December 1978. These techniques are generally easy to perform, do not require expensive supplies or laboratory facilities, and offer high levels of selectivity. However, these methods are slow. Substrate-based assays are hindered by the requirement to first grow or cultivate pure cultures of the targeted organism, which can take twenty-four hours or longer. This time constraint severely limits the effectiveness to provide rapid response to the presence of virulent strains of microorganisms.

Other molecular biology techniques that have received a great deal of attention recently are Polymerase Chain Reaction (PCR) methods. PCR detection of specific microorganisms in a sample involves extraction of the genetic material (RNA and/or DNA) in a sample, amplification of a target genetic sequence specific to the microorganism of interest, and then detection of the amplified genetic material. PCR techniques in principal can provide high selectivity owing to the uniqueness of the detected genetic material, high sensitivity because of the substantial amplification of the target genetic material, and rapid results owing to the potentially fast amplification process, though to date PCR instruments have not delivered the hoped-for sensitivity or specificity. Moreover, PCR instruments and reagents are quite expensive and highly trained technicians are needed to perform the tests.

Some attempts have been made to improve upon substrate-based classical bacterial detection methods using bacteriophage amplification. Bacteriophage are viruses that have evolved in nature to use bacteria as a means of replicating themselves. A bacteriophage (or phage) does this by attaching itself to a bacterium and injecting its genetic material into that bacterium, inducing it to replicate the phage from tens to thousands of times. Some bacteriophage, called lytic bacteriophage, rupture the host bacterium releasing the progeny phage into the environment to seek out other bacteria. The total incubation time for phage infection of a bacterium, phage multiplication (amplification) in the bacterium, and release of the progeny phage after lysis can take as little as an hour depending on the phage, the bacterium, and the environmental conditions. Microbiologists have isolated and characterized over 5,000 phage species, including many that specifically target bacteria at the species or even the strain level. United States Patent Application Publication No. 2002/0127547 discloses a method of detecting bacterial strains that immobilizes bacteriophage on a support in which the sample to be tested is contacted to the support, using the specificity of bacteriophage to bacteria to attach the bacteria to the bacteriophage. The bound bacteria and bacteriophage are then separated from the sample, and various methods are suggested for detecting the presence of the target bacteria. However, the method has not been commercially successful, probably because the phage plates disclosed do not store well and take a very long time to prepare fresh ones before each use. In addition, a very large number of bacteria are required to provide a signal level high enough to be measured. In fact, the one example given in the application, which used a known laboratory culture of bacteria, says only that colorimetric reaction that was supposed to detect proteins bound to the phage or the phage proteins “was followed for several (2-5) hours”, and no results are provided.

Various other references that have disclosed the use of bacteriophage to enhance the speed and efficacy of bacterial infection include U.S. Pat. No. 5,985,596 issued Nov. 16, 1999 to Stuart Mark Wilson, U.S. Pat. No. 6,461,833 B1 issued Oct. 8, 2002 to Stuart Mark Wilson, U.S. Pat. No. 4,861,709 issued Aug. 29, 1989 to Ulitzur et al., U.S. Pat. No. 5,824,468 issued Oct. 20, 1998 to Scherer et al., U.S. Pat. No. 5,656,424 issued Aug. 12, 1997 to Jurgensen et al., U.S. Pat. No. 6,300,061 B1 issued Oct. 9, 2001 to Jacobs, Jr. et al., U.S. Pat. No. 6,555,312 B1 issued Apr. 29, 2003 to Hiroshi Nakayama, U.S. Pat. No. 6,544,729 B2 issued Apr. 8, 2003 to Sayler et al., U.S. Pat. No. 5,888,725 issued Mar. 30, 1999 to Michael F. Sanders, U.S. Pat. No. 6,436,661 B1 issued Aug. 20, 2002 to Adams et al., U.S. Pat. No. 5,498,525 issued Mar. 12, 1996 to Rees et al., U.S. Patent Application Publication No. 2004/0137430 published Jul. 15, 2004 in the name of Dwight L. Anderson et al., U.S. Patent Application Publication No. 2002/0127547 published Sep. 12, 2002 in the name of Stefan Miller, and U.S. Patent Application Publication No. 2004/0224359 published Nov. 11, 2004 in the name of Angelo J. Madonna et al.

U.S. Pat. No. 6,265,169 issued Jul. 24, 2001 to Riccardo Cortese et al. and United States Patent Application Publication No. 2004/0121403 published Jun. 24, 2004 in the name of Steven L. Highlander et al. disclose the use of bacteriophage to isolate bacteria or other molecules, and the Cortese patent also discloses the use of PCR to amplify bacteriophage DNA. However, neither of these involves bacteriophage amplification.

All of these bacteriophage approaches in theory offer faster results than do traditional microbiology methods. However, all of them also have some downside, such as the need for complex preparation processes and/or the use of complex processes and/or expensive equipment to perform the detection analysis. Thus, although bacteriophage amplification has been studied for more than a decade and significant research investments in the technology have been made by many organizations, as evidenced by the above prior art, no commercially effective process for detection of bacteria via bacteriophage amplification has yet been developed. What is needed is a detection method combining the sensitivity, simplicity, and/or low cost of substrate-based assays with the rapid results offered by bacteriophage amplification.

### SUMMARY OF THE INVENTION

The invention solves the above problems, as well as other problems of the prior art, by providing methods and apparatus for detecting living microorganisms using the principle of phage amplification combined with the use of a bacteriophage progeny biological substance selected from bacteriophage nucleic acid (DNA/RNA), bacteriophage protein, and bacteriophage intermediate protein, as an indicator of the presence of the microorganism. Here, bacteriophage intermediate protein is protein that is created in the bacteriophage replication process that does not become part of the replicated bacteriophage. Bacteriophage nucleic acid can include nucleic acid injected by the parent bacteriophage or nucleic acid formed in the replication process. This provides a much larger multiplication factor and higher signal, which permits the detection of smaller concentrations of bacteria. Preferably, the invention includes amplifying the bacteriophage progeny biological substance. If the progeny biological substance is nucleic acid, the amplification is preferably selected from PCR, SDA, and Rolling Circle.

In some embodiments, the invention solves the above problems, as well as other problems, by dissociating or lysing the infected bacteria prior to the completion of the bacteriophage replication cycle in combination with an assay for a biological substance associated with the bacteriophage, such as nucleic acid or phage protein, which speeds up the assay and also provides a much larger multiplication factor and higher signal, which permits the detection of smaller concentrations of bacteria.

In other embodiments, the invention solves the above problems, as well as other problems, by determining a reference level of a bacteriophage nucleic acid, assaying a test level of said bacteriophage nucleic acid in said infected sample, and comparing said test level to said reference level to determine the presence or absence of said target microorganisms in said sample. This provides a higher signal level by both subtracting out the background noise and using a form of detection that has a higher multiplication factor and can be amplified.

In some embodiments, the invention also solves the above problems, as well as other problems, by mobilizing the microphage, either by coupling the bacteriophage to mobile substrates, or by not coupling the phage to a substrate at all, both of which permit more thorough contact of a sample, particularly insitu samples, to the parent bacteriophage, thereby increasing the signal derived from samples with small numbers of the target microorganisms.

The invention provides a method of detecting the presence or absence of a target microorganism in a sample to be tested, the method comprising: (a) combining with the sample, parent bacteriophage capable of infecting the target microorganism to create a bacteriophage exposed sample; (b) providing conditions to the bacteriophage exposed sample sufficient to allow the bacteriophage to infect the target microorganism; (c) dissociating or lysing the microorganism prior the completion of the progeny bacteriophage replication process; and (d) assaying the bacteriophage exposed sample for a bacteriophage progeny biological substance to determine the presence or absence of the target microorganism in the sample. Preferably, the target microorganism is a bacterium and the assaying comprises detecting the bacteriophage progeny biological substance as an indication of the presence of the target bacterium in the sample. Preferably, the bacteriophage progeny biological substance is a protein. Preferably, the bacteriophage progeny biological substance is a nucleic acid. Preferably, the assaying comprises amplifying the nucleic acid using a nucleic acid amplification method. Preferably, the nucleic acid amplification method is selected from the group consisting of PCR, SDA, and Rolling Circle. Preferably, the method further comprises isolating the bacteriophage progeny biological substance from the sample prior to the assaying. Preferably, the assaying comprises a process selected from the group consisting of gel electrophoresis, oligo capture, fluorescence labeling, and colorimetric assays. Preferably, the sample is an insitu sample having an insitu matrix, the method further comprising isolating the microorganism from the insitu matrix prior to the assaying. Preferably, the isolating of the microorganism is performed prior to the combining. Preferably, the isolating comprises capturing the microorganism and removing it from the matrix. Preferably, the capturing comprises phage mediated capturing. Preferably, the method further comprises cleaning the captured microorganism after the removing. Preferably, the method further comprises placing the captured and cleaned microorganism in a sub-sample reagent. Preferably, the method further comprises measuring a reference level of the bacteriophage progeny biological substance, and the assaying comprises measuring a test level of the bacteriophage progeny biological substance and comparing the test level to the reference level. Preferably, the reference level is measured for a sample in which the target microorganism is known not to be present. Preferably, the method further comprises furnishing a first portion of the sample and a second portion of the sample; and killing the microorganism in the first portion of the sample; and wherein the reference level is measured for the first portion of the sample and the test level is measured for the second portion of the sample. Preferably, the measuring of a reference level comprises taking a reference sub-sample from the bacteriophage exposed sample and performing a reference assay on the reference sub-sample before any bacteriophage amplification has occurred in the reference sub-sample, and wherein the test level is measured for the bacteriophage exposed sample after some of the bacteriophage replication process has occurred. Preferably, the combining comprises tagging the parent bacteriophage. Preferably, the assaying comprises isolating the tagged parent bacteriophage from the bacteriophage exposed sample. Preferably, the tagging comprises attaching the parent bacteriophage to a mobile substrate that can be added to or retrieved from the sample. Preferably, the isolating comprises removing the tagged, parent bacteriophage from the sample.

The invention also provides a method of detecting the presence or absence of a target microorganism in a test sample, the method comprising: (a) combining with the microorganism an amount of parent bacteriophage capable of infecting the target microorganism to create a bacteriophage exposed sample; (b) determining a reference level of a bacteriophage nucleic acid; (c) providing conditions to the combined microorganism and parent bacteriophage sufficient to allow the parent bacteriophage to infect the target microorganism to create an additional amount of the nucleic acid; and (d) assaying a test level of the bacteriophage nucleic acid in the infected sample and comparing the test level to the reference level to determine the presence or absence of the target microorganism in the sample. Preferably, the microorganism is a bacterium and the assaying comprises detecting the nucleic acid as an indication of the presence of the target bacterium in the sample. Preferably, the assaying comprises a process selected from the group consisting of gel electrophoresis, oligo capture, fluorescence labeling, and calorimetric assays. Preferably, the assaying also comprises amplifying the bacteriophage nucleic acid using an amplification method selected from the group consisting of PCR, SDA, or Rolling Circle. Preferably, the determining comprises: providing a reference sample in which it is known that the target microorganism is not present; adding the parent bacteriophage to the reference sample; providing the conditions to the reference sample; and assaying the reference sample to provide the reference level. Preferably, the determining comprises: furnishing a reference portion of the sample; killing the microorganism in the reference portion; adding the parent bacteriophage to the reference sample; providing the conditions to the reference sample; and assaying the reference sample to provide the reference level. Preferably, the determining comprises a taking of a reference sub-sample from the bacteriophage exposed sample and performing a reference assay on the reference sub-sample before any bacteriophage amplification has occurred in the reference sub-sample.

The invention further provides a method of detecting the presence or absence of a target microorganism in a sample to be tested, the method comprising: (a) combining with the sample, bacteriophage capable of infecting the target microorganism to create a bacteriophage exposed sample; (b) providing conditions to the bacteriophage exposed sample sufficient to allow the bacteriophage to infect the target microorganism to create intermediate proteins associated with the replication of the bacteriophage, which intermediate proteins are not present in the fully replicated bacteriophage; and (c) assaying the intermediate proteins to determine the presence or absence of the target microorganism. Preferably, the target microorganism is a bacterium and the assaying comprises detecting the intermediate protein as an indication of the presence of the target bacterium in the sample. Preferably, the providing comprises dissociating the target microorganism prior to the completion of the replication cycle of the bacteriophage. Preferably, the sample is an insitu sample having an insitu matrix, the method further comprising isolating the microorganism from the insitu matrix prior to the assaying. Preferably, the isolating of the microorganism is performed prior to the combining. Preferably, the isolating comprises capturing the microorganism and removing it from the matrix. Preferably, the capturing comprises phage mediated capturing.

In a further aspect, the invention provides a method of determining the presence or absence of a target microorganism in a sample to be tested, the method comprising: (a) combining with the microorganism, an amount of parent bacteriophage capable of infecting the target microorganism to create a bacteriophage exposed sample; (b) capturing the microorganism in the sample without using a bacteriophage or bacteriophage protein; (c) providing conditions to the combined microorganism and parent bacteriophage sufficient to allow the bacteriophage to infect the target microorganism to create injected bacteriophage nucleic acid, additional bacteriophage nucleic acid, or additional bacteriophage protein; and (d) assaying the injected bacteriophage nucleic acid, the additional bacteriophage nucleic acid, or the additional bacteriophage protein to determine the presence or absence of the target microorganism in the insitu sample. Preferably, the microorganism is a bacterium and the assaying comprises detecting the injected bacteriophage nucleic acid, the additional bacteriophage nucleic acid, or the additional bacteriophage protein as an indication of the presence of the target bacterium in the sample. Preferably, the assaying comprises a process selected from the group consisting of gel electrophoresis, oligo capture, fluorescence labeling, and colorimetric assays. Preferably, the assaying is of the injected bacteriophage nucleic acid or the additional bacteriophage nucleic acid, and the assaying further comprises amplifying the injected bacteriophage nucleic acid or the additional bacteriophage nucleic acid using an amplification method selected from the group consisting of PCR, SDA, or Rolling Circle. Preferably, the method further comprises dissociating or lysing the microorganism prior to the completion of the replication cycle of the bacteriophage. Preferably, the capturing comprises immunomagnetic separation. Preferably, the capturing comprises utilizing an antibody to capture the microorganism. Preferably, the capturing comprises removing the microorganism from the sample. Preferably, the method further comprises cleaning the microorganism after the removing. Preferably, the method also comprises placing the cleaned microorganism in a sub-sample environment.

In yet another aspect, the invention provides a method of detecting the presence or absence of a target microorganism in a test sample, the method comprising: (a) combining with the microorganism, an amount of parent bacteriophage capable of infecting the target microorganism to create a bacteriophage exposed sample; (b) providing conditions to the combined microorganism and parent bacteriophage sufficient to allow the parent bacteriophage to infect the target microorganism to create an additional amount of the nucleic acid; (c) assaying a test level of the bacteriophage nucleic acid in the infected sample and comparing the test level to the reference level to determine the presence or absence of the target microorganism in the sample; and (d) wherein the assaying comprises amplifying the bacteriophage nucleic acid using an amplification method selected from the group consisting of PCR, SDA, or Rolling Circle. Preferably, the assaying comprises a process selected from the group consisting of gel electrophoresis, oligo capture, fluorescence labeling, and calorimetric assays.

In yet a further aspect, the invention provides a method of detecting the presence or absence of a target microorganism in a sample to be tested, the method comprising: (a) combining with the sample, bacteriophage that are not immobilized on a support, the bacteriophage capable of infecting the target microorganism to create a bacteriophage exposed sample; (b) providing conditions to the bacteriophage exposed sample sufficient to allow the bacteriophage to infect the target microorganism to create injected bacteriophage nucleic acid or additional bacteriophage nucleic acid; and (c) assaying the injected bacteriophage nucleic acid or the additional bacteriophage nucleic acid to determine the presence or absence of the target microorganism.

The above summary is intended to illustrate some examples of the objects, features, and advantages of the invention so that the invention can be better understood. In some embodiments of the invention, only one of the above objects may be realized, and in others a plurality of such objects may be realized. However, the above objects are intended to be exemplary, not all inclusive, so there will be instances in which none of the above objects are realized in a particular embodiment. For example, the methods and apparati of the invention can be used for detecting microorganisms other than bacteria, such as fungi, mycoplasmas, protozoa, and other microscopic living organisms. Thus, if the word “bacteria” in the above objects is replaced with the more general term “microorganism”, valid objects of the invention are expressed. Numerous other features, objects, and advantages of the invention will become apparent from the following description when read in conjunction with the accompanying drawings.

### BRIEF DESCRIPTION OF THE DRAWINGS

FIG. 1 illustrates a simple embodiment of the invention in which phage nucleic acid or phage protein is assayed to detect bacteria;

FIG. 2 illustrates the incubation process of phage infection, amplification, and cell lysis;

FIG. 3 illustrates the preferred process of the invention in more detail;

FIG. 4 is a side cross-sectional view of a lateral flow device;

FIG. 5 is an illustration of a bacteriophage;

FIG. 6 illustrates a second embodiment of the invention wherein phage are added to the sample to give an initial concentration below the detection limit and where the phage are dissociated such that phage subcomponent biomarkers are detected;

FIG. 7 illustrates a third embodiment wherein tagged phages are added to the sample;

FIG. 8 illustrates a fourth embodiment of the invention wherein tagged parent phage are added to the sample and where the progeny phage are dissociated such that phage subcomponent biomarkers are detected;

FIG. 9 illustrates detection of antibiotic resistant bacteria using the invention;

FIG. 10 illustrates a phage amplification process; and

FIG. 11 illustrates an exemplary embodiment of an assay process according to the invention utilizing immunomagnetic separation and bacteriophage nucleic acid detection.

### DETAILED DESCRIPTION OF THE PREFERRED EMBODIMENT

In the preferred procedure, bacteriophage specific to a species of bacteria is added to a suspension suspected of containing the targeted bacterium. If this bacterium is present, the bacteriophage infects the cell and replicates numerous copies of itself. Lysing of the bacterium, either naturally or through the use of a lysing agent, releases the replicated phages into the surrounding medium. The use of a bacteriophage nucleic acid (DNA/RNA) as a marker permits gel electrophoresis, oligo capture, fluorescence labeling, and other nucleic acid detection systems to be used to detect the bacteria.

For many microorganism detection applications, the samples that potentially contain the target microorganism(s) are complex organic mixtures containing large numbers of proteins, enzymes, lipids, nucleic acids, etc. One such application is detecting bacterial contamination in foods such as raw beef or chicken where the sample may contain ground up meat. Such samples are referred to herein as insitu samples, and the matrixes, such as ground meat, in such samples are referred to as insitu matrixes. Insitu is a Latin word meaning “in the natural state”. This word is conventionally used in science to distinguish such natural environments from controlled laboratory environments. In this usage, it distinguishes the natural environments in which microorganisms to be detected are found from laboratory broths, serums, growth substrates, culture media, and similar environments that are expressly tailored to provide a controlled environment for biochemistry. The term insitu sample includes both pure natural state samples as well as samples that include a diluted fraction of a natural state sample. As long as any of the original insitu matrix is included, the sample is an insitu sample.

The method of the invention relies on the bacteriophage, or simply phage, amplification to detect the presence of a target microscopic living organism (microorganism), such as a bacterium, in a sample. In this disclosure, the terms “bacteriophage” and “phage” include bacteriophage, phage, mycobacteriophage (such as for TB and paraTB), mycophage (such as for fungi), mycoplasma phage or mycoplasmal phage, and any other term that refers to a virus that can invade living bacteria, fungi, mycoplasmas, protozoa, and other microscopic living organisms and uses them to replicate itself. Here, “microscopic” means that the largest dimension is one millimeter or less. Bacteriophage are viruses that have evolved in nature to use bacteria as a means of replicating themselves. A phage does this by attaching itself to a bacterium and injecting its DNA into that bacterium, inducing it to replicate the phage hundreds or even thousands of times. Some bacteriophage, called lytic bacteriophage, rupture the host bacterium, releasing the progeny phage into the environment to seek out other bacteria. The total incubation time for phage infection of a bacterium, phage multiplication or amplification in the bacterium, to lysing of the bacterium takes anywhere from tens of minutes to hours, depending on the phage and bacterium in question and the environmental conditions.

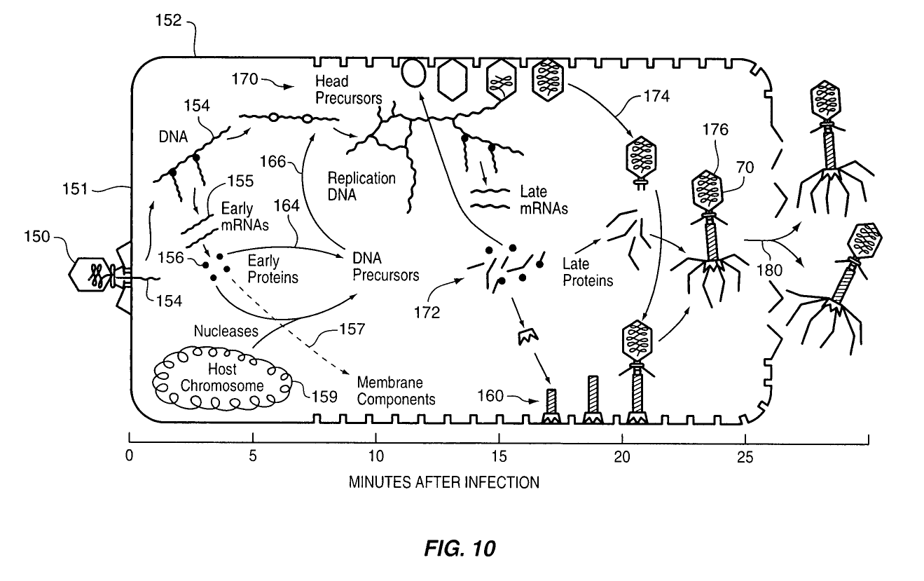

The disclosed detection method offers a combination of specificity, sensitivity, simplicity, speed, and/or cost which is superior to any currently known microscopic organism detection method. The method taught herein relies on the usage of bacteriophage nucleic acid or bacteriophage protein to indirectly detect the presence of one or more target bacterium in a sample. A typical bacteriophage 70, in this case MS2-E. coli is shown in FIG. 5. Structurally, a bacteriophage 70 comprises a protein shell or capsid 72, sometimes referred to as a head, that encapsulates the viral nucleic acids 74, i.e., the DNA and/or RNA. A bacteriophage may also include internal proteins 75, a neck 76, a tail sheath 77, tail fibers 78, an end plate 79, and pins 80. The capsid 72 is constructed from repeating copies of one or more proteins. Referring to FIG. 10, when a phage 150 infects a bacterium 152, it attaches itself to a particular site on the bacterial wall or membrane 151 and injects its nucleic acid 154 into that bacterium, inducing it to replicate the phage from tens to thousands of copies. The process is shown in schematic in FIG. 10. The DNA evolves to early mRNAS 155 and early proteins 156, some of which become membrane components along line 157 and others of which utilize bacteria nucleases from host chromosomes 159 to become DNA precursors along line 164. Others migrate along the direction 170 to become head precursors that incorporate the DNA along line 166. The membrane components evolve along the path 160 to form the sheath, end plate, and pins. Other proteins evolve along path 172 to form the tail fibers. When formed, the head releases from the membrane 151 and joins the tail sheath along path 174, and then the tail sheath and head join the tail fibers at 176 to form the bacteriophage 70. The early proteins 156, precursor proteins along paths 172 and 174, and any other protein that is formed during the process of replication but is not present in the parent or final replicated bacteriophage is referred to herein as intermediate proteins. As we shall see below, the phage nucleic acid, the intermediate proteins, and the phage proteins associated with the progeny phage are particularly useful as indicators of the presence of the target microorganism. Some bacteriophage, called lytic bacteriophage, rupture the host bacterium, shown at 180, releasing the progeny phage into the environment to seek out other bacteria. Lytic phages are typically used in the method disclosed herein. However, non-lytic phages can be used, particularly if they or the bacteria can be activated to release progeny phage or portions of progeny phage after the progeny phage infect the host bacteria.

The total cycle time for phage infection of a bacterium, phage multiplication or amplification in the bacterium, to lysing of the bacterium takes anywhere from minutes to hours, depending on the phage and bacterium in question and the environmental conditions. As an example, the MS2 bacteriophage infects strains of Escherichia coli and is able to produce 10,000 copies to 20,000 copies of itself within 40 minutes after attachment to the target cell. The capsid of the MS2 phage comprises 180 copies of an identical protein. This means that for each E. coli infected by MS2, upwards of 1.8×10⁶ individual capsid proteins are produced. The process of phage infection whereby a large number of phage and an even larger number of capsid proteins are produced for each infection event is called phage amplification.

Microbiologists have isolated and characterized many thousands of phage species, including specific phages for most human bacterial pathogens. Individual bacteriophage species exist that infect bacterial families, individual species, or even specific strains. Table 1 lists some such phages and the bacterium they infect.

TABLE 1

| PHAGE | BACTERIAL TARGET |
|---|---|
| MS2 | E. coli, Enterococci |
| φA1122 | Yersinia pestis |
| CSLEC0157 | E. coli 0157 |
| ΦFelix 0-1 | Salmonella spp. |
| Chp1 | Chlamydia trachomatis |
| Gamma | B. anthracis |
| A511 | Listeria spp. |

This invention takes advantage of the existing characteristics of bacteriophage, such as highly specific phage-bacterial infection, phage amplification, and short incubation time, resulting in a bacterial detection method which is highly specific to target bacteria, very sensitive, fast, simple to perform, and/or can be quite economical. Moreover, unlike other phage-based bacterial detection methods, the preferred method described herein uses phages that are not genetically modified to include bioreporter or inducer genes. This dramatically reduces the time and costs associated with developing specific bacterial tests utilizing this method.

3. Detailed Description

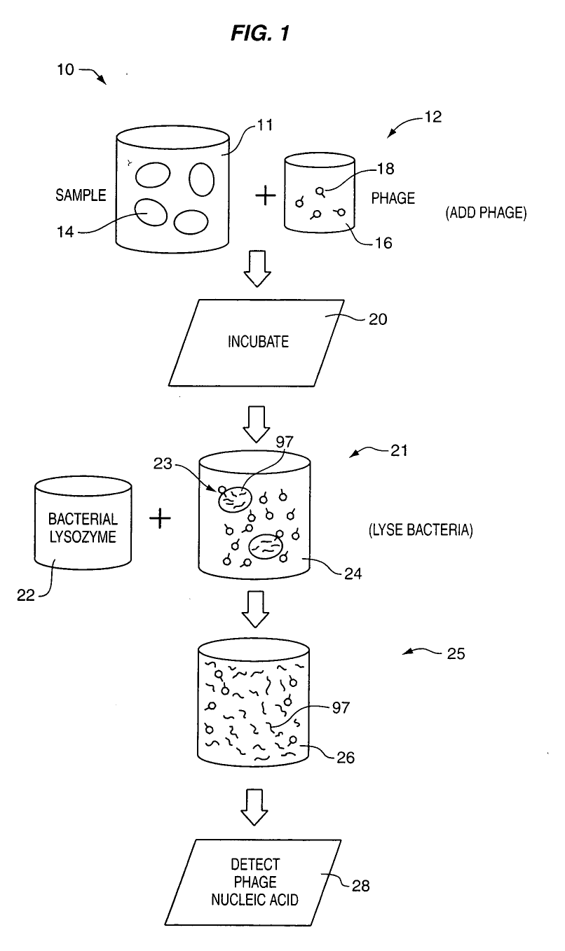

FIG. 1 illustrates a first embodiment 10 of the method to detect specific bacteria in a sample. In a first ‘ADD PHAGE’ process 12, parent bacteriophage 18 that will infect the target bacterium 14 is combined with the raw sample 11 of bacterium 14. In the preferred embodiment, the bacteriophage, preferably in a 20 suspension or solution 16, is added in a predetermined concentration to the raw sample 11 of bacterium 14. Here, the term “raw sample” refers to the sample prior to the addition of the phage, and it may or may not be an insitu sample. The raw sample/phage combination is referred to herein as “the test sample 24” or the “bacteriophage exposed sample 24”. If the object of the method is to detect a specific bacterium at the species or strain level, then a correspondingly specific phage is used in the method. For example, the φA1122 phage can be used to specifically detect Y. Pestis. Conversely, a less specific phage can be used to detect a wider range of bacteria in a sample. The phage MS2 will infect many different E. coli species as well as Enterococci and, thus, is quite suitable for detecting fecal contamination in water.

To detect multiple bacteria, one species of bacteriophage is added to the raw sample for each target bacterium giving a single test sample that contains all of the target bacteria and associated phages. For the purposes of simplicity, the method will be described henceforth as it applies to detecting a single bacterium. It should be clear to those skilled in the art how each process of the method can be performed simultaneously with one test sample utilizing unique bacterium/phage combinations to detect each target bacterium.

The raw sample 11 containing the target bacterium 14 is generally in a liquid form but could be a solid or a powder. The raw sample could be a mixture or suspension containing many different organic and inorganic compounds. It may have been pretreated in a variety of ways to prepare it for testing. For example, the raw sample may have been purified or filtered to remove unwanted components or to concentrate the target bacterium. It may have been cultured in a media conducive to the incubation of the target bacterium or to induce the target bacterium into a more viable state. The raw sample may be in a relatively untreated state such as might be the case with a sputum, blood, or water sample. It should be clear to one skilled in the art that pretest sample preparation may include any one of a wide variety of suitable processes, and the raw sample may take many different forms.

The phage itself may be added to the sample in a variety of forms. It may be added in a dry state. The phage may be mixed or suspended into a liquid reagent mixture. It may be suspended in a vial to which the raw sample is added. It also may take any other suitable form. The phage added to the raw sample is herein referred to as “the parent phage”.

Returning to FIG. 1, the test sample 24 is incubated, preferably for a predetermined time. For this method, the test sample should preferably be in a condition that is conducive to phage infection of the target bacterium prior to the incubation process. This can be accomplished in a variety of ways well known to those skilled in the art. For example, the parent phage may be mixed into a reagent that, when added to the raw sample, results in a test sample conducive to infection. The test sample may be prepared in many different ways to establish conditions conducive to phage infection.

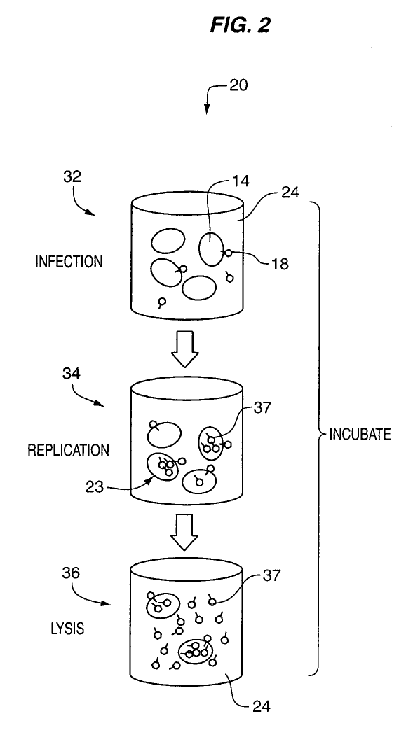

The INCUBATE process 20 is shown in FIG. 2. The parent phage 18 infects 32 the target bacteria 14 by attaching themselves to cell walls of the target bacteria and injecting the viral nucleic acid to create infected bacteria 23. Replication 34 of progeny phage as indicated in FIG. 10 then proceeds within the host bacteria. In some embodiments, the replication of progeny page is permitted to proceed to completion. If lytic phages are used, the host ruptures in a lysis process 36 releasing the progeny phage 37 into the test sample where they may infect other target bacteria. This incubation process may proceed for one or more cycles of infection, amplification, and lysis. Assuming there were target bacteria in the raw sample, the test sample will contain a large number of progeny phage for each individual bacterium infected during the incubation process. In this embodiment, as will be discussed below, the progeny phage are lysed to release the phage nucleic acid.

Returning to FIG. 1, in the preferred embodiment, a bacterial lysome 22 for the particular microorganism is added in process 21, preferably prior to complete formation of the progeny phage, which, in process 25, causes the cell walls of essentially all the particular microorganism, such as a bacterium, present in the test sample 24 to rupture, thereby releasing essentially all bacteriophage progeny biological substances 97, including nucleic acid injected into the bacterium by the parent bacteriophage, bacteriophage intermediate proteins, progeny bacteriophage nucleic acid, and progeny bacteriophage phage proteins, and also all microorganism internal biological materials, including microorganism DNA, contained therein. For example, this releases up to tens of thousands of phage DNA. It should be noted that the term “bacteriophage progeny biological substances” includes nucleic acid 154 injected into the microorganism by the parent phage, but does not include complete progeny bacteriophage.

When necessary to distinguish the LYSE step from the natural or passive lysing caused by the bacteriophage, we will refer to it as “actively lysing” herein. The usage of the microbial, or more specifically, bacterial, lysozyme in combination with nucleic acid detection shortens the time required to carry out the method taught herein. For slowly incubating phage, this can make a substantial difference. For the purposes of this invention, the term “actively lysing” shall refer to any material, apparatus, or process by which the microorganism host is induced to rupture, thus releasing the progeny phage material into the test sample, including, but not limited to, chemical means such as traditional lysozymes, chloroform, or acid treatments, or a physical process, such as changing the osmotic pressure.

Process 28, DETECT PHAGE NUCLEIC ACID, of the embodiment illustrated in FIG. 1 comprises detecting a nucleic acid or protein associated with the phage. If progeny phage nucleic acid or progeny phage protein is detected, it is an indirect indicator of the presence of the target bacteria in the raw sample. For the embodiment of the method described thus far, the parent phage added to the raw sample and the progeny phage, if produced during the incubation process, are identical. This means that, even if there are no target bacteria in the test sample, there will still be phage present during detection process 28 that might give rise to an associated background signal. Generally, since phage are much more resistant to lysing agents than bacteria, phage nucleic acid should not be present. However, it may be possible that one or more phage may lyse due to natural defects in phage. A method of solving this problem is to control the initial concentration of parent phage in the test sample such that the background signal due to any phage nucleic acid produced is undetectable in detection process 28. Thus, if no target bacteria are present in the test sample such that no phage amplification occurs, then no signal is detected at the end of the test. A higher concentration of parent phage can still be used, provided that any background signal can be distinguished from the signal arising from the parent plus progeny phage. The lowest bacterial concentration at which the resulting signal in process 28 can be distinguished from the background signal represents the sensitivity limit of the method. A simple way of doing this is to perform a test on a reference sample in which it is known that no bacteria are present. This creates a reference result which can be compared with a corresponding result from a test sample. If the test result from the test sample clearly shows a higher signal level than the reference sample, then the presence of the target bacteria is indicated.

Another way to reduce the background signal is to detect one or more intermediate proteins in the process 28. Generally, these intermediate proteins are distinguishable from the proteins of the parent phage. Thus, the parent phage do not provide a background signal for these intermediate proteins.

Any detection method or apparatus that detects phage nucleic acid or phage protein will suffice for this method 28. Preferred methods for detection of phage nucleic acid are gel electrophoresis, oligo capture, fluorescence labeling, colorimetric methods, and other nucleic acid detection systems. Preferred methods for detection of phage proteins include immunoassay methods utilizing antibody-binding events to produce detectable signals, ELISA, flow cytometry, western blots, aptamer-based assays, radioimmunoassay, immunofluoresence, lateral flow immunochromatography (LFI), matrix-assisted laser desorption/ionization with time-of-flight mass spectrometry (MALDI-TOF-MS), referred to herein as MALDI, the use of a SILAS surface which changes color as a detection indicator, and other protein detection methods.

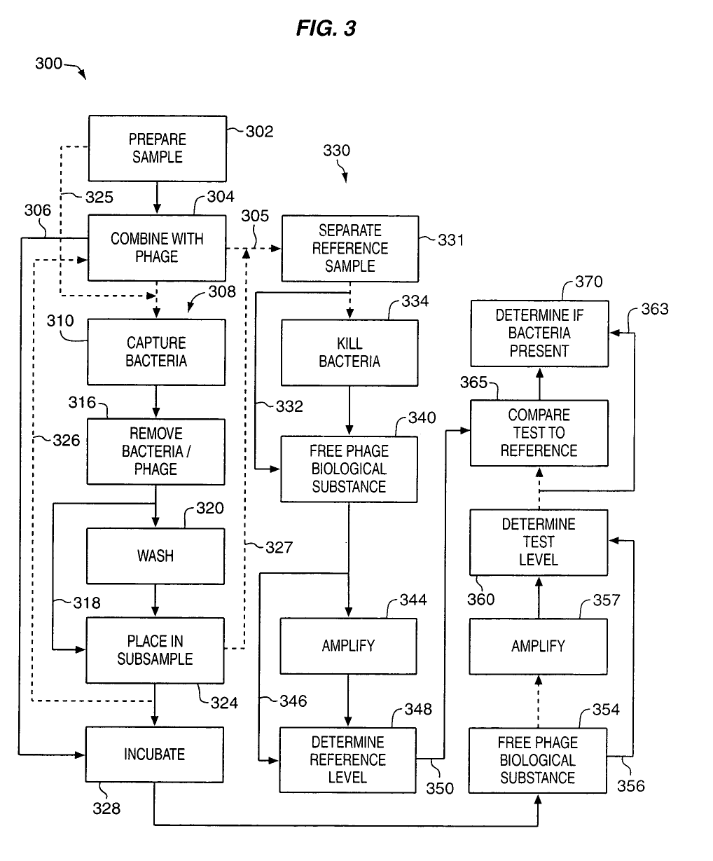

FIG. 3 is a flow chart illustrating the preferred processes 300 of the invention, including preferred optional processes, in more detail. In this flow chart, the paths leading to optional processes, such as path 305, are shown by dashed lines. The process 300 begins with the preparation of a sample at 302. Usually this will involve the providing of a raw sample to be tested, which sample will usually include complex insitu matrixes. Generally, a portion of the raw sample will be diluted with a liquid such as sterile water to provide a suitable volume of prepared insitu sample. This sample is then combined at 304 with a suitable quantity of bacteriophage. A feature of the invention is that a very large quantity of bacteriophage, such as 10⁶ to 10¹⁰ bacteriophage, may be added while still providing an accurate result. In a more preferred embodiment, 10⁷ to 10⁹ bacteriophage may be added. In the most preferred embodiment, from 5×10⁷ to 5×10⁸ bacteriophage are added. This large quantity of bacteriophage greatly accelerates the process. Generally, these large quantity of bacteriophage may be added in processes in which the reference process 330 is used, or processes in which the parent phage is isolated from the progeny phage, or in processes in which the microorganism but not the phage is dissociated. The phage may be added by combining a solution of phage with the sample solution, combining a phage containing substrate with the sample, or any other convenient method of adding the phage to the sample or adding the sample to the phage.

The process then preferably goes directly to subprocess 328 via path 306, which subprocess 328 will be discussed below, or, optionally, it goes to an isolation, purification, and/or concentration process 308. The isolation, purification, and/or concentration process 308 begins with process 310 in which the bacteria is captured. Process 310 preferably comprises capturing the microorganism with either the phage which were added in process 304, or with a non-phage capturing medium, such as antibodies. In the former case, the phage that are combined in process 304 are preferably attached to a substrate, which may be a plate or other large surface or structure, or may be a probe made of mesh, magnetic beads, or other mobile structure that can be stirred in the sample prepared in step 302. In the latter case, the non-bacteriophage capturing medium may be attached to magnetic beads, a large surface area structure, or a probe made of mesh or other mobile structure. In any case, the capturing medium is left in contact with the microorganism for a suitable length of time, such as several minutes, for the capture process to take place. In either case, the bacteria/phage combination is removed from the insitu sample in process 316, optionally washed one or more times in process 320, and then placed in a sub-sample environment in process 324. The sub-sample environment is preferably a liquid medium in which the bacteria will grow normally. The sample is then incubated in process 328, which comprises optimum conditions for infection of the microorganism by the bacteriophage and replication of the bacteriophage, which conditions vary depending on the microorganism and bacteriophage and are known in the art.

An optional process is illustrated by dashed lines 325 and 326. In this process, the isolation, purification, and/or concentration process 308 takes place prior to the combining process 304. The capture process 310 takes place after the sample preparation process 302 as shown by path 325. Then, after the Place In Sub-Sample Process 324, the combining process 304 is performed, as indicated by path 326. This optional process then proceeds directly to the incubate process 328 via path 306.

The process 300 next proceeds to process 354 in which the bacteriophage biological substances, which will include one or more of injected DNA, intermediate proteins, additional bacteriophage nucleic acid, and additional bacteriophage proteins, are freed from the microorganism, and optionally, from the bacteriophage. Here, additional means biological substances which are produced in the replication process; that is, they are not part of the parent bacteriophage. The freeing may be by dissociation or lysing of the microorganism, or by some other method of causing the microorganism to disgorge the biological substances, such as osmotic shock. If the process 300 is one in which the bacteriophage replication is permitted to proceed to completion, the freeing process will also include a process in which the progeny bacteriophage are dissociated into their component nucleic acids and proteins. However, preferably, incubation process 328 is shorter than the bacteriophage replication cycle and process 354 takes place before the bacteriophage replication cycle can be completed. If the bacteriophage progeny biological substance is a nucleic acid, the freed bacteriophage biological substances may be amplified in process 357, or the process 300 may proceed directly to subprocess 360 via path 356. Generally, the amplification process 357 is a nucleic acid amplification process, such as PCR, SDA, or Rolling Circle. After amplification, the process 300 proceeds to subprocess 360. If the bacteriophage progeny biological substance is a protein, the process 300 proceeds directly to subprocess 360 via path 356. Subprocess 360 comprises a determination of the level of the selected bacteriophage biological substance in the test sample. If the bacteriophage biological substance is a nucleic acid, the subprocess 360 preferably comprises a gel electrophoresis, oligo capture, fluorescence labeling, or calorimetric assay. It also may comprise any other nucleic acid detection systems or process. If the bacteriophage biological substance is a protein, the subprocess 360 may be selected from immunoassay methods utilizing antibody-binding events, ELISA, flow cytometry, western blots, aptamer-based assays, radioimmunoassay, immunofluoresence, and lateral flow immunochromatography (LFI). Other protein detection methods are matrix-assisted laser desorption/ionization with time-of-flight mass spectrometry (MALDI-TOF-MS), referred to herein as MALDI, and the use of a SILAS surface which changes color as a detection indicator, or any other protein detection process. The process 300 then proceeds directly to process 370, or optionally, to process 365.

Returning to process 304, in an optional embodiment of the invention, after the sample is combined with bacteriophage, a reference process 330 is initiated as shown by path 305. In process 330, a reference sample is separated from the test sample in process 331. If the optional isolation, purification, and/or concentration process 308 is performed, and it is also desired to include the reference process 330, then the reference sample is separated after process 324, as shown by path 327. Process 331 generally comprises transferring a suitable portion of the test sample, preferably half of it, to a separate container or other medium. The process 300 may then proceed directly to process 338 via path 332, or, optionally, proceed to process 334. In process 334, the microorganisms are killed, which may be by adding a suitable agent, such as an antibiotic, by heating, or by any other suitable method. The phage biological substances are then freed, preferably by the same method as used in process 354. If the biological substance was amplified in process 357, the reference sample is also amplified in process 344, preferably by the same method as used in process 357. If not, the reference process 330 proceeds to process 348 via path 346. In process 348, the level of the selected bacteriophage biological substance in the reference sample is determined, preferably by the same method as used in process 360. The test level result found in subprocess 360 is then compared to the reference level result found in process 350. The process 300 then proceeds to process 370 in which the presence of the microorganism is determined. Generally, if the test level result is essentially the same as the reference level result, or they are both within an empirically predetermined range, process 370 finds that the target microorganism is not present. However, if the test level result shows more than a predetermined level of the specified bacteriophage biological substance, the process 370 finds that the microorganism is present. Process 370 may be performed automatically by test equipment pursuant to a predetermined algorithm, or may be performed manually.

It should be understood that FIG. 3 does not specifically show every variation of the invention. The various processes and subprocesses may in certain instances be performed in a different order, and additional processes and subprocesses may be added. For example, the capture bacteria process may be performed prior to the combining process 304, and then proceed directly to the incubate process 328 without the other subprocesses in the isolation, purification, and/or concentration process 308. The purpose of FIG. 3 is to illustrate and discuss each of the important subprocesses of the invention, and illustrate the preferred order of execution and the more important optional pathways. It is not intended to be exhaustive.

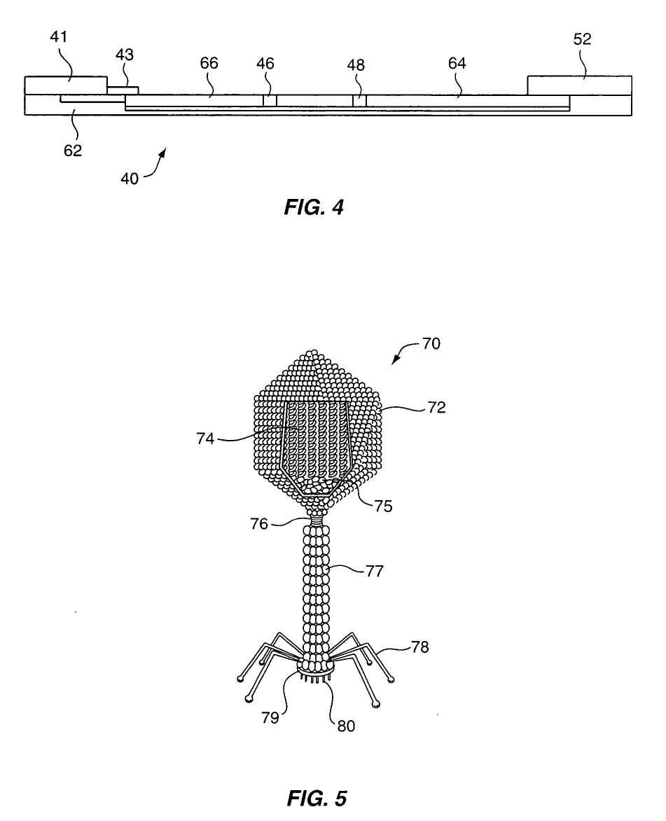

FIG. 4 illustrates how LFI can be implemented with a lateral flow strip 40 to detect the presence of phage in a test sample. For a more detailed description of this process, see U.S. patent application Ser. No. 10/823,294 referenced above. A cross-sectional view of the lateral flow strip 40 is shown in FIG. 4. The lateral flow strip 40 preferably includes a sample application pad 41, a conjugate pad 43, a substrate 64 in which a detection line 46 and an internal control line 48 are formed, and an absorbent pad 52, all mounted on a backing 62, which preferably is plastic. The substrate 64 is preferably a porous mesh or membrane. It is made by forming lines 43, 46, and optionally line 48, on a long sheet of said substrate, then cutting the substrate in a direction perpendicular to the lines to form a plurality of substrates 64. The conjugate pad 43 contains colored beads, each of which has been conjugated to a first antibody forming first antibody-bead conjugates. First antibody selectively binds to the phage protein in the test sample. Detection line 46 and control line 48 are both reagent lines and each form an immobilization zone; that is, they contain a material that interacts in an appropriate way with the bacteriophage protein. In the preferred embodiment, the interaction is one that immobilizes the bacteriophage protein. Detection line 46 comprises immobilized second antibodies, with antibody line 46 perpendicular to the direction of flow along the strip, and being dense enough to capture a significant portion of the phage protein in the flow. Second antibody also binds specifically to the phage protein. First antibody and second antibody may or may not be identical. Either may be polyclonal or monoclonal antibodies. Optionally, strip 40 may include a second reagent line 48 including a third antibody. Third antibody may or may not be identical to one or more of the first and second antibodies. Second reagent line 48 may serve as an internal control zone to test if the assay functioned properly.

One or more drops of a test sample 50 are added to the sample pad. The test sample flows along the lateral flow strip 40 toward the absorbent pad 52 at the opposite end of the strip. As the phage protein particles flow along the conjugate pad toward the membrane, they pick up one or more of the first antibody-bead conjugates forming protein-bead complexes. As the protein-bead complexes move over row 46 of second antibodies, they form an immobilized and concentrated first antibody-bead-protein-second antibody complex. If enough protein-bead complexes bind to the row 46 of immobilized second antibodies, a colored line becomes visible to the naked eye. A visible line indicates that the target bacteria were present in the raw sample. If no line is formed, then target bacteria were not present in the raw sample or were present in concentrations too low to be detected with the lateral flow strip 40. An important aspect of the invention is that this test works reliably even if the concentration of parent phage added to the raw sample is high. The antibody-bead conjugates are color moderators that are designed to interact with the bacteriophage protein. When they are immobilized in the immobilization zone 46, they cause the immobilization zone to change color.

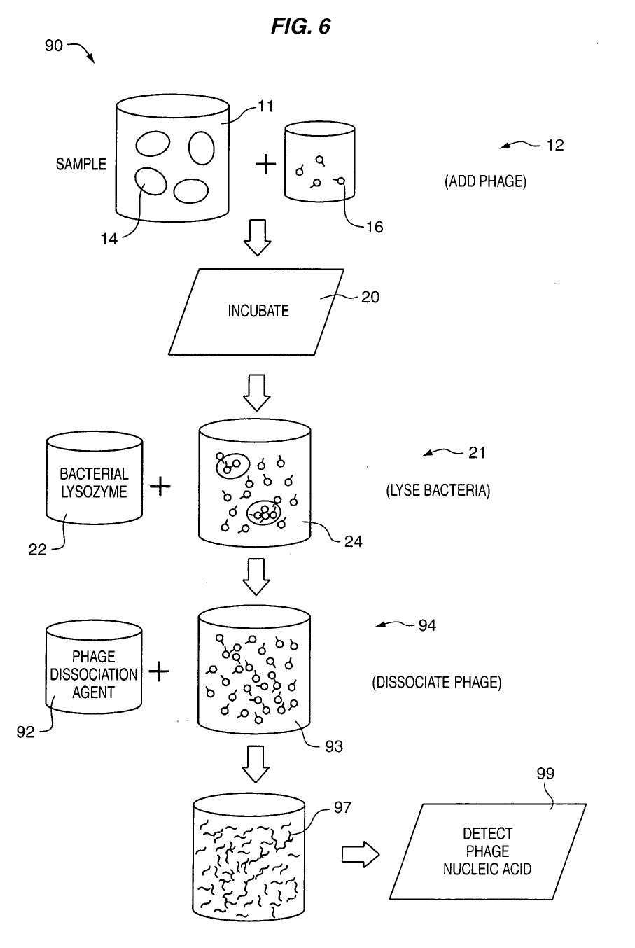

FIG. 6 illustrates a second embodiment 90 of a method to detect target bacteria according to the invention, which method 90 has enhanced sensitivity. Processes 12, 20, and optional process 21 consisting of ADD PHAGE, INCUBATE, and LYSE BACTERIA are identical to the corresponding processes described in association with FIGS. 1 and 2, though it should be understood that the full incubation process of FIG. 2 is preferred here. After optional process 21, the test sample contains an abundance of phage nucleic acid and phage protein particles if target bacteria were present in the raw sample.

As shown in FIG. 6, process 94 of the second embodiment 90, DISSOCIATE PHAGE, comprises adding a phage dissociation agent 92 to the test sample. The phage dissociation agent 92 breaks up the phage particles into their constituent components 97, including individual capsid proteins and viral nucleic acids. Examples of phage dissociation agents are acid treatments, urea, denaturing agents, and enzymes. Any suitable phage dissociation agent may be used. In this process, a dissociated bacteriophage substance 97 is produced.

Process 99 of the embodiment illustrated in FIG. 6, DETECT PHAGE NUCLEIC ACID, comprises detecting a biomarker, i.e., dissociated bacteriophage nucleic acid 97 associated with the dissociated phage subcomponents. With respect to the foregoing discussion, it should be understood that a bacteriophage substance can be both a dissociated bacteriophage substance and at the same time be associated with the bacteriophage. That is, the phrase “a dissociated bacteriophage substance” means a substance that is no longer a part of a whole bacteriophage, while the term “associated with the bacteriophage” means that substance was at one time a part of a bacteriophage or is produced in the process of bacteriophage infection or replication. Owing to the usage of the phage dissociation agent in process 94, there are an abundance of individual nucleic acids 97 that can be detected in process 99. As with the first embodiment, these can be detected using established antigen-antibody based immunoassay techniques. In addition, the exposed viral genetic material can be detected with other established techniques including PCR, genetic probe biosensors, photoaptamers, molecular beacons, or gel electrophoresis. Any appropriate phage nucleic acid detection method or apparatus may be used.

Keeping the concentration of parent phage in the test sample below the background detection limit makes for a very simple test method: add phage to the raw sample, incubate, and then detect phage biomarkers. However, there is a potential disadvantage as well. The potentially low concentration of parent phage may result in conditions where the ratio of parent phage to target bacteria in the test sample is less than 1; i.e., the Multiplicity Of Infection (MOI) is low. To ensure that all target bacteria in the test sample have a high probability of being infected, the incubation time in Process 20 can be made, for example, a time equivalent to two or more cycles of infection and lyses. Thus, test simplicity is offset by potentially longer testing times. This potential limitation can be overcome if the signal associated with the parent phage can be eliminated or significantly reduced such that higher concentrations of parent phage can be utilized—MOls greater than 5. It can also be overcome if the signal due to the progeny phage is enhanced, such as by the use of genetically enhanced phage, both of which are discussed in detail herein.

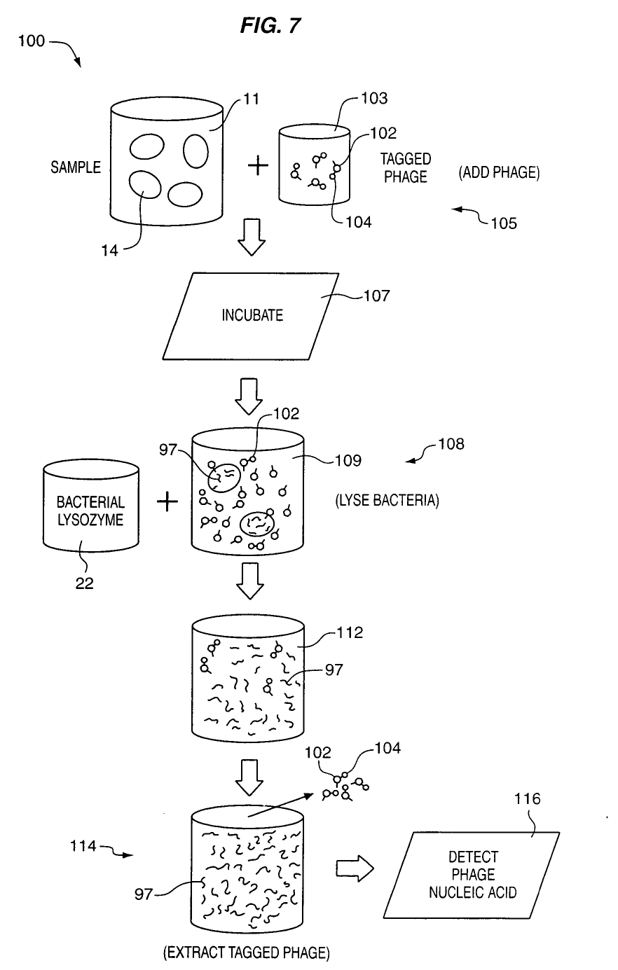

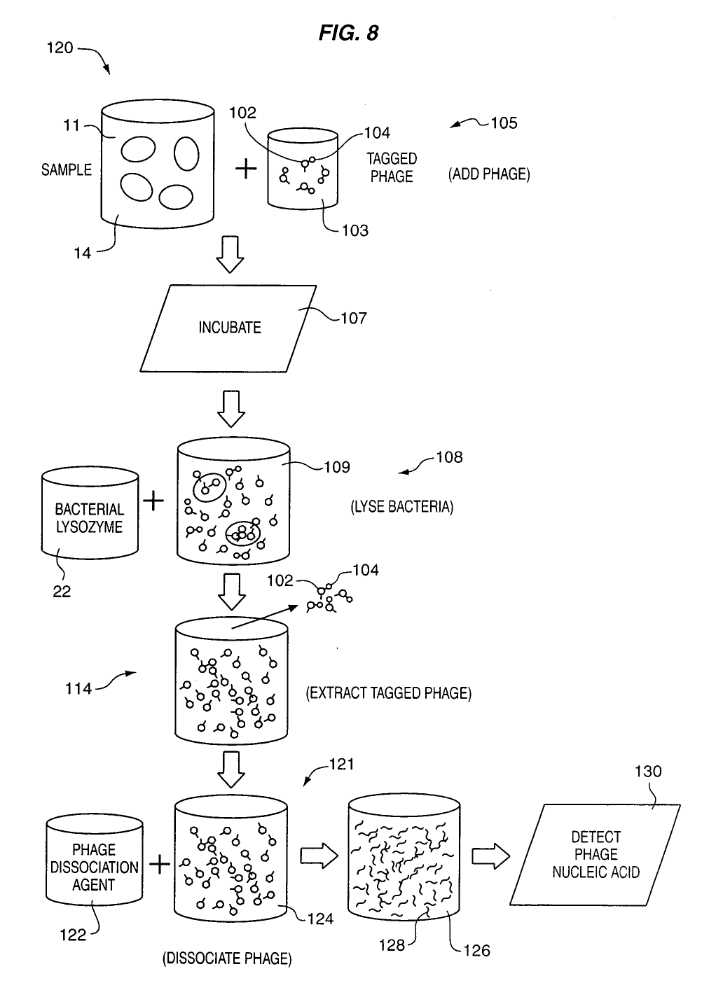

FIG. 7 illustrates a third embodiment 100 of the inventive method to detect target bacteria in a sample wherein more rapid results are achievable. In this embodiment of the invention, the parent phage 102 that are combined with the raw sample are tagged, indicated by a tag symbol at 104, such that they can be subsequently removed from the test sample, isolated from the portion of the test sample in which the bacteriophage are detected, or otherwise neutralized prior to analysis such that primarily untagged progeny phage contribute to the detected signal. For example, in one embodiment a biotinylated phage was used as a parent phage. Biotinylated bacteriophage are strongly attracted to streptavidin. This strong affinity was used to subsequently segregate the tagged parent phage from the test sample, as discussed below. The tagged parent phage can also be attached to a physical substrate. Preferably, the substrate is a mobile substrate, such as a bead, probe, or mesh structure which can be easily moved in the solution. The tagged bacteriophage then can be segregated from the progeny bacteriophage by removing the substrate from the test sample or by detecting the bacteriophage in a portion of the test sample that is segregated from the substrate.

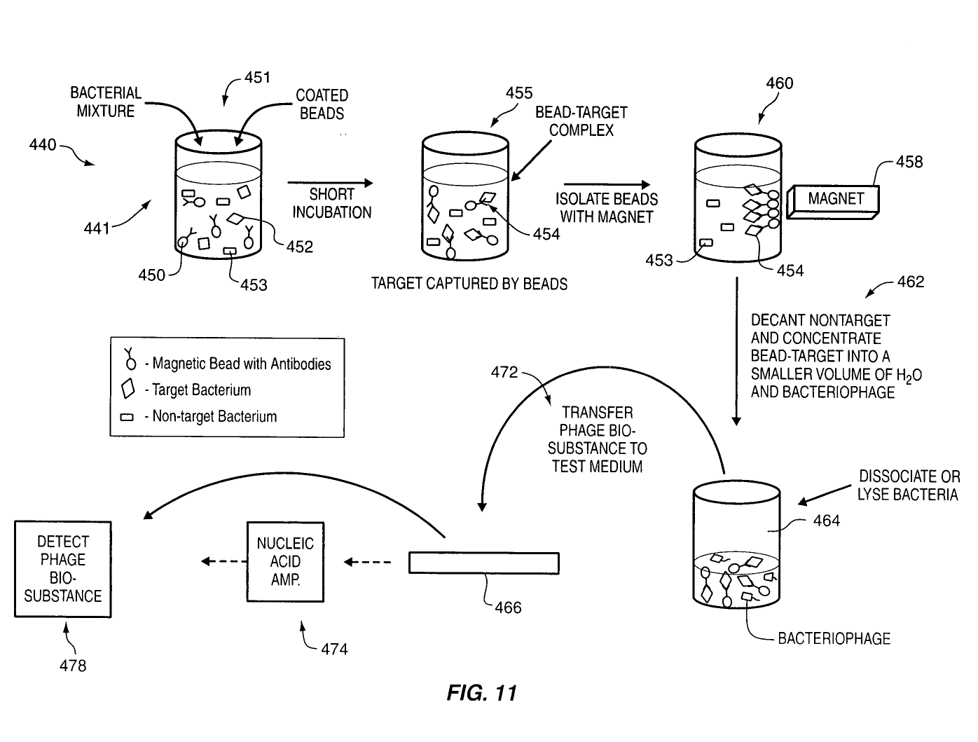

Processes 105, 107, and 108, ADD PHAGE, INCUBATE, and LYSE BACTERIA, respectively, of this embodiment are the same as processes 12, 20, and 21, respectively, of FIGS. 1 and 6, with the exception that the solution of parent phage 103 added to the raw sample in Process 105 contains tagged phage 102, so bacteriophage exposed sample 109 contains both tagged phage 102 external of the bacteria and phage nucleic acid 97 within the bacteria. Thus, the lysed solution 112 will contain both tagged phage and phage nucleic acid. In process 114, EXTRACT TAGGED PHAGE, the tagged parent phage are segregated from the progeny bacteriophage by extracting or substantially removing them from the test sample or otherwise isolating the parent phage from the progeny phage such that they cannot interfere with or contribute to the analyzed signal. If the tagged parent phages are attached to a physical substrate when added to the raw sample in process 105, then the substrate and associated parent phage are preferably physically removed from the test sample in process 114. Biotinylated phage that are not attached to a physical substrate also can be readily segregated or removed from the test sample. In one embodiment, streptavidin-coated magnetic beads were added to the test sample where they rapidly collected the biotinylated parent phage. A magnet was then used to aggregate and remove the magnetic beads along with the bound parent phage from the test sample. See the discussion of magnetic extraction associated with FIG. 11 below. Similarly, in another embodiment, a streptavidin-coated mesh was stirred through the test sample, gathering up essentially all of the biotinylated parent phage from the test sample. Other physical substrates or probes other than a mesh can also be used. In another embodiment, a lateral flow device was used. A portion 66 (FIG. 4) of the mesh substrate 64 prior to the antibody strip 46 was impregnated with streptavidin, coating the mesh fibers. The streptavidin-coated mesh gathered up and immobilized the tagged parent phage by binding the parent phage to the portion 66 before they reached the antibody strip 46. The progeny phage did not bind to the streptavidin and, thus, flowed freely down the strip and were visually detected. Similarly, other portions of the lateral flow device could be coated or impregnated with streptavidin, such as the sample pad 41 onto which the test sample is dropped.

The method described herein is not limited to these examples of tagging parent phage and subsequently removing them from or segregating them within the test sample. Other parent phage tagging/phage segregation methods will be readily apparent to those skilled in the art.

Process 116 of the embodiment illustrated in FIG. 7, DETECT PHAGE NUCLEIC ACID, is to analyze the test sample to detect nucleic acid injected by the parent phage and nucleic acid associated with the forming progeny phage as a surrogate marker for target bacteria present in the raw sample. The detection means used with this embodiment are identical to those described with respect to processes 28 and 29 of the embodiments 10 and 90, respectively, as illustrated in FIGS. 1 and 6, respectively. As with the earlier embodiments, any suitable detection method or apparatus may be used.

FIG. 8 illustrates a fourth embodiment 120 of a method to detect target bacteria in a sample, in which method 120 the sensitivity is enhanced. Embodiment 120 is a combination of the methods taught in embodiments 90 and 100. Processes 105, 107, 108, and 114 are identical to those taught with embodiment 100 and illustrated in FIG. 7, i.e., ADD PHAGE, INCUBATE, optionally LYSE BACTERIA, and EXTRACT TAGGED PHAGE, respectively. In this embodiment, as in the embodiment of FIG. 6, the phage replication is preferably permitted to go to completion in the INCUBATE step. Specifically, embodiment 120 incorporates tagged parent phage in process 105 and a parent phage removal or segregation process in process 114.

In process 121, DISSOClATE PHAGE, a phage dissociation agent 122 is added to the test sample 124 as taught in process 94 of embodiment 90 and illustrated in FIG. 6. In a preferred embodiment, the tagged parent phage is physically removed from the test sample in process 114 rather than simply segregated so that it will not be exposed to the phage dissociation agent in process 121. Thus, the test sample 124 contains only progeny phage, and the dissociated test sample 126 will contain biological marker material, including phage nucleic acid, only from progeny phage. In this manner, the amplification associated with dissociating the phage will combine with the phage amplification of process 107, resulting in a much higher total amplification. For example, if the phage amplification process gives an amplification of 1000 per bacterium and the phage has 10,000 copies of a particular nucleic acid, then the combined amplification will be 10³×10⁴ or 10⁷ per target bacteria infected in the test sample. If the parent phage is not removed, then the total amplification is only the phage amplification that occurs in process 107; i.e. 10³, because the amplification arising from dissociating the phage will occur to both the parent phage and to the progeny phage, thus canceling out the second amplification process.

DETECT PHAGE NUCLEIC ACID process 130 of the embodiment 120 illustrated in FIG. 6 is preferably the same as any of the processes 28, 99, and 116 of the earlier embodiments.

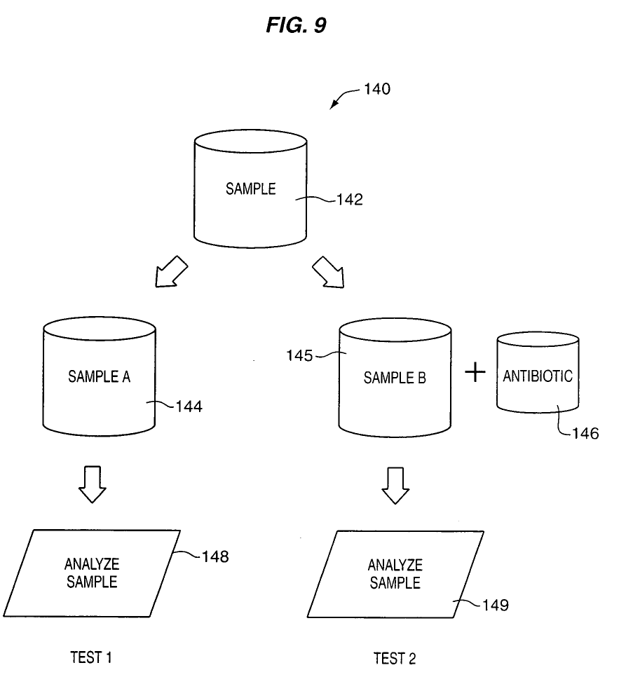

FIG. 9 illustrates a method 140 by which any of the embodiments of the invention can be used to detect a target bacterium, and if present, determine if it is resistant to one or more antibiotics. A sample 142 that may contain the target bacterium is divided into two: a first Sample A, indicated by 144, and a second Sample B, indicated by 145. A first antibiotic 146 is added to Sample B whereupon the target bacteria in Sample B are killed if they are not resistant to the first antibiotic. Samples A and B are then analyzed at 148 and 149 to detect the presence of viable target bacteria in each, giving Result A and Result B. Any of the methods taught in this invention can be used for these analyses. If Result A is positive, it indicates that the target bacterium is present in the original sample. If Result B is also positive, it indicates that the target bacterium is resistant to the first antibiotic. If, on the other hand, Result B is negative, then the target bacterium is not resistant to the first antibiotic. To screen for antibiotic resistance to any one of a range of antibiotics simultaneously, then all of the antibiotics of interest are added to Sample B prior to analyzing for the target bacterium. If the target bacterium is detected in both the pure sample and the antibiotic treated sample, it indicates that the target bacterium in the sample is resistant to one of the added antibiotics. This process can also be used to determine the susceptibility of bacteria to antibiotics or other decontaminants. It can also be used to test whether a bacterial decontamination process has been successful. By dividing a sample into a control portion and a test portion, the effectiveness of bacteriological methods and materials can be tested. Those skilled in the art will recognize that the processes of the invention can be used in nearly every instance where it is desirable to determine if live bacteria are present.

The process of FIG. 9 also illustrates another process according to the invention. In this case, the antibiotic 146 represents any substance or method that will kill bacteria, such as heating or an antibacterial agent. Since the bacteria in sample B are known to be dead, bacteriophage will not replicate and create progeny nucleic acid. Thus, sample B should give a null result. Thus, any difference between the result 148 and the result 149 will indicate the presence of the target microorganism.

FIG. 11 illustrates an exemplary embodiment of an assay process 440 according to the invention utilizing immunomagnetic separation in combination with nucleic acid or protein detection. A sample 451 includes biological targets 452 and other biological materials 453. In process 441, beads 450 coated with an antibody that is specific to the target 452 are added to the sample 451, which is stirred or otherwise incubated for a short time, such as twenty minutes, to form bead-target complexes 454 in process 455. A magnet 458 is used to isolate the bead-target complexes 454 in process 460. The non-target remainder is decanted 462, and the bead-target complexes are concentrated into a smaller volume of solvent such as water with added target-specific bacteriophage to form concentrated sample 464. The phage and target are incubated for a short time, and the target is then dissociated or lysed to release phage nucleic acid, protein, or other bacteriophage progeny biological substance. The lysing is active lysing but may also be natural lysing. A drop of the water with bacteriophage progeny biological substance is then transferred in process 472 to suitable test medium 466 which may be a solid, a fluid, or a device, such as a flow strip, which can be utilized for the desired detection process. Optionally, if the biological substance is nucleic acid, it may be amplified at 474 using PCR, SDA, Rolling Circle, etc. The bacteriophage progeny biological substance is then detected in process 478.

Still referring to FIG. 11, in an alternative process 441, the beads 450 may be coated with a target-specific bacteriophage. In this case, the incubation is kept very short, for example 1 to 10 minutes, while the beads are stirred vigorously in the bacterial mixture. In this embodiment, additional bacteriophage in the solvent in process 464 is not necessary. However, in this case, the entire process from the addition of the beads in process 441 to the dissociation of lysing of the target in process 464 should be short, preferably no more than 20 minutes. However, if the cycle time from infection to lysing of the particular bacteriophage strain is longer, this time can be longer also.

In both the processes just described, the beads may be replaced by a mesh or other substrate that can be stirred in bacterial mixture so that the antibodies or bacteriophage can be kept mobile and be exposed to a significant portion of the mixture during the incubation time. The mobility of the antibodies or bacteriophage, whether on the beads, mesh, or other mobile substrate, greatly speeds up this step and makes the process practical.

The protocol described herein is intended to reduce, if not eliminate, the need for nucleic acid amplification techniques such as PCR, SDA, Rolling Circle, etc. The concentration of bacteriophage nucleic acid after amplification according to the invention is often great enough to be detected under most conventional detection systems without additional amplification using PCR, SDA, Rolling Circle, etc. In such cases, the protocol will reduce costs significantly, and be less affected by temperature, cross contamination, probe sequence errors, and complex extraction techniques. The nucleic acid or protein being detected is that of the infecting bacteriophage and represents an amplification factor of up to 20,000 copies per infected pathogen. Upon cell lysis, each of the bacteriophage is capable of re-infecting the sample, adding further sensitivity to the system and a significant improvement in signal-to-noise ratio. Since most pathogens known in nature have unique bacteriophage infectives, each with unique nucleic acid and protein sequences, this protocol offers a precise, accurate, sensitive, and selective method for pathogen detection. The system allows for simultaneous detection of multiple pathogens due to the uniqueness of each of the bacteriophage sequences.

When additional amplification of the phage nucleic acids is required to make it detectable, conventional PCR methods can be used. This two-stage amplification protocol has significant advantages over direct, single-step PCR amplification of the bacterial nucleic acids. First, far fewer PCR amplification steps are required to detect the bacteria, resulting in significantly reduced measurement error. If phage replication amplifies the number of phage by 1000, then 10 PCR cycles will be required to give a DNA count in excess of 1 million per initial bacterium in the sample. If only PCR is used to amplify the bacterial nucleic acids, 20 amplification cycles are required. Because PCR methods potentially amplify non-specific nucleic acids in a sample, an additional 10 amplification cycles result in 1000 times more non-specific nucleic acid signal than does the two-step phage-amplification/PCR protocol. The contaminating nucleic acids result in significantly higher rates of false positives. In effect, the signal-to-noise ratio is many times higher with the two-step phage-amplification/PCR protocol than with the one-step PCR protocol. The higher signal-to-noise ratio has the added benefit of lower detection limits for the bacterium of interest.

An additional advantage of the two-step amplification protocol is enhanced selectivity. Bacteriophage infection of a target bacterium is a highly selective process when a suitably selective bacteriophage is used. The phage selectivity enhances the selectivity of the nucleic acid identification process used at the end of the two-step amplification protocol.

The two-step amplification protocol also has the advantage of distinguishing between viable and non-viable bacteria, whereas the one-step PCR method does not. A sample containing non-viable bacteria could still give a positive result in a conventional PCR test, because the nucleic acid from non-viable bacteria are amplified as readily as those of viable bacteria. A sample containing only non-viable bacteria will give a negative result using the two-stage protocol because phage amplification only occurs in viable bacteria.

Thus, the two-step amplification protocol consisting of a phage amplification step followed by PCR amplification of the phage nucleic acids is more sensitive, selective, and can distinguish between viable and non-viable bacteria.

For many microorganism detection applications, the samples that potentially contain the target microorganism(s) are complex organic mixtures containing large numbers of proteins, enzymes, lipids, nucleic acids, etc. One such application is detecting bacterial contamination in foods such as raw beef or chicken where the sample may contain ground up meat. Detecting specific bacterial nucleic acids in such organically complex samples is challenging. It is highly advantageous to isolate the nucleic acids from such a matrix prior to amplification and detection. Suitably modified, the phage nucleic acid amplification method can be used to isolate the desired nucleic acids from a complex organic matrix that may contain target microorganism(s). An example of such a process follows.

1) First, the parent bacteriophage is tagged in such a way that the parent phage can be readily added to or retrieved from the sample. This can be done in a number of ways including: a) biotinylating the parent bacteriophage and binding them to small magnetic beads; or b) biotinylating the parent phage and chemically binding it to a substrate such as a probe or a porous substrate. The tagging must be done in such a manner that the bacteriophage can still readily bind to and infect the target microorganism.

2) The tagged, parent bacteriophage then is added to the sample and allowed to incubate sufficiently long enough to allow some or all of the target microorganisms in the sample to bind to tagged, parent bacteriophage but not long enough for the bacteriophage infection cycle to be completed, resulting in lyses of the target microorganisms. Thus, if it takes 90 minutes for a bacteriophage to bind to a target microorganism, infect it, replicate within it, then lyse the microorganism; then, for the purposes of this method, the tagged, parent bacteriophage would be added to the sample for a period of time less than 90 minutes. This second process may be considered a capture process where the tagged, parent bacteriophage are used to capture some or all of the target microorganisms in the sample.

3) The tagged, parent bacteriophage are then retrieved and removed from the sample. This process will also serve to remove the captured target microorganisms from the sample. If the tagged bacteriophage are bound to magnetic beads, then a magnet can be used to segregate and remove the tagged bacteriophage (and bound microorganisms) from the sample. The usage of magnetic beads to separate desired species from a complex mixture is called ImmunoMagnetic Separation (IMS). If the tagged bacteriophage are bound to a substrate, then the substrate is removed or isolated from the remainder of the sample.

4) Optionally, the tagged, parent bacteriophage are cleaned off to remove remaining unwanted traces from the sample. This cleaning process must be performed carefully such that neither the parent bacteriophage nor target microorganisms bound to them are lost.

5) After retrieval and optional cleaning, the tagged, parent phage and bound microorganisms may be added to a reagent solution and allowed to incubate sufficiently long enough to complete the bacteriophage infection cycle, thereby releasing progeny phage into the reagent. The reagent solution used herein should be conducive to continued growth of the captured target microorganisms but should not contain compounds that would contaminate subsequent assay processes. In particular, the solution should be essentially sterile such that it contains no nucleic acids other than that added during this process.

6) Optionally, the tagged, parent bacteriophage are removed from the reagent solution. This removal process results in the removal of all parent bacteriophage from the reagent solution leaving only progeny bacteriophage that are released from infected target microorganisms.

7) Optionally, the nucleic acid in the reagent solution may be amplified using conventional amplification methods such as PCR. If the optional cleaning process above has been performed, the reagent solution will essentially contain nucleic acids from parent and progeny bacteriophage and from the lysed target microorganisms captured in process 2 above. If optional process 6 above is also performed, then the only nucleic acids present in the reagent solution will be that of progeny bacteriophage and of captured, target microorganisms.

8) The reagent sample is then assayed to determine the presence or absence of bacteriophage associated nucleic acid as an indication of the presence or absence of the target microorganism in the original sample.

The advantages of the above assay method are several. First, bacteriophage are used to capture and isolate the target microorganisms from the complex sample matrix, thereby eliminating many problems associated with PCR assays of raw samples. Second, removing the tagged, parent bacteriophage from the reagent solution eliminates virtually all of the background DNA signal that would arise from the parent bacteriophage in subsequent analysis processes. This makes it possible to use large numbers of parent bacteriophage to capture target microorganisms in the original sample without compromising the ultimate sensitivity of the assay.

Optionally, a background reference assay may be performed by taking a reference sub-sample from the bacteriophage exposed sample and assaying that reference sub-sample before any bacteriophage amplification has occurred in the reference sub-sample. The reference result then can be obtained from this optional assay. Provided the assay methods and conditions used with the reference sub-sample and the bacteriophage exposed sample are identical, then the reference assay result is a direct measure of the background signal associated with the bacteriophage exposed sample. Any detectable increase in the signal for the assay performed on the bacteriophage exposed sample as compared to the reference assay result is a direct indication of the presence of the target microorganism in the original sample.

U.S. patent application Ser. No. 10/823,294, which has been incorporated herein by reference, discloses many aspects of the bacteriophage amplification and detection process, which can be combined with the processes disclosed herein. However, in the process of the present application, instead of detecting the capsid or other proteins, the detection method used is the detection of a bacteriophage biological substance, preferably nucleic acid (DNA/RNA), as a marker. This permits the use of gel electrophoresis, oligo capture, fluorescence labeling, and other nucleic acid detection systems to detect the presence of a pathogen. As a result, the process of the invention is relatively sensitive, fast, simple, and/or economical as compared to the prior art.

There has been described a microorganism detection method which is specific to a selected organism, sensitive, simple, fast, and/or economical, and having numerous novel features. The invention can be used in a wide variety of applications including human clinical diagnostics, veterinary diagnostics, food pathogen detection, environmental testing, and other detection. It should be understood that the particular embodiments shown in the drawings and described within this specification are for purposes of example and should not be construed to limit the invention, which will be described in the claims below. Further, it is evident that those skilled in the art may now make numerous uses and modifications of the specific embodiment described, without departing from the inventive concepts. Equivalent structures and processes may be substituted for the various structures and processes described; the subprocesses of the inventive method may, in some instances, be performed in a different order; or a variety of different materials and elements may be used. Consequently, the invention is to be construed as embracing each and every novel feature and novel combination of features present in and/or possessed by microorganism detection apparatus and methods described.

1. A method of detecting the presence or absence of a target microorganism in a sample to be tested, said method comprising:
(a) combining with said sample, parent bacteriophage capable of infecting said target microorganism to create a bacteriophage exposed sample;
(b) providing conditions to said bacteriophage exposed sample sufficient to allow said bacteriophage to infect said target microorganism;
(c) dissociating or lysing said microorganism prior to the completion of the progeny bacteriophage replication process; and
(d) assaying said bacteriophage exposed sample for a bacteriophage progeny biological substance to determine the presence or absence of said target microorganism in said sample.

2. A method as in claim 1 wherein said target microorganism is a bacterium and said assaying comprises detecting said bacteriophage progeny biological substance as an indication of the presence of said target bacterium in said sample.

3. A method as in claim 1 wherein said bacteriophage progeny biological substance is a protein.

4. A method as in claim 1 wherein said bacteriophage progeny biological substance is a nucleic acid.

5. A method as in claim 4 and wherein said assaying comprises amplifying said nucleic acid using a nucleic acid amplification method.

6. A method as in claim 5 wherein said nucleic acid amplification method is selected from the group consisting of PCR, SDA, and Rolling Circle.

7. A method as in claim 1 and further including isolating said bacteriophage progeny biological substance from said sample prior to said assaying.

8. A method as in claim 1 wherein said assaying comprises a process selected from the group consisting of gel electrophoresis, oligo capture, fluorescence labeling, and calorimetric assays.

9. A method as in claim 1 wherein said sample is an insitu sample having an insitu matrix, said method further comprising isolating said microorganism from said insitu matrix prior to said assaying.

10. A method as in claim 9 wherein said isolating of said microorganism is performed prior to said combining.

11. A method as in claim 9 wherein said isolating comprises capturing said microorganism and removing it from said matrix.

12. A method as in claim 11 wherein said capturing comprises phage mediated capturing.

13. A method as in claim 11 and further comprising cleaning said captured microorganism after said removing.

14. A method as in claim 13 and further comprising placing said captured and cleaned microorganism in a sub-sample reagent.

15. A method as in claim 14 wherein said combining is performed after said captured and cleaned microorganism is placed in said sub-sample reagent.

16. A method as in claim 1 and further including measuring a reference level of said bacteriophage progeny biological substance, and said assaying comprises measuring a test level of said bacteriophage progeny biological substance and comparing said test level to said reference level.

17. A method as in claim 16 wherein said reference level is measured for a sample in which said target microorganism is known not to be present.

18. A method as in claim 17 and further comprising;
furnishing a first portion of said sample and a second portion of said sample; and
killing said microorganism in said first portion of said sample;
and wherein said reference level is measured for said first portion of said sample and said test level is measured for said second portion of said sample.

19. A method as in claim 16 wherein said measuring of a reference level comprises taking a reference sub-sample from said bacteriophage exposed sample and performing a reference assay on said reference sub-sample before any bacteriophage amplification has occurred in said reference sub-sample, and wherein said test level is measured for said bacteriophage exposed sample after some of the bacteriophage replication process has occurred.

20. A method as in claim 16 wherein said microorganism is a bacterium, and said assaying comprises detecting bacteriophage nucleic acid as an indication of the presence of said target bacterium in said sample.

21. A method as in claim 1 wherein said combining comprises tagging said parent bacteriophage.

22. A method as in claim 21 wherein said assaying comprises isolating said tagged parent bacteriophage from said bacteriophage exposed sample.

23. A method as in claim 22 wherein said tagging comprises attaching said parent bacteriophage to a mobile substrate that can be added to or retrieved from said sample.

24. A method as in claim 22 wherein said isolating comprises removing said tagged, parent bacteriophage from the sample.

25. A method as in claim 24 and further comprising cleaning said tagged, parent bacteriophage to remove remaining unwanted traces of said insitu matrix.

26. A method of detecting the presence or absence of a target microorganism in a test sample, said method comprising:
(a) combining with said microorganism, an amount of parent bacteriophage capable of infecting said target microorganism to create a bacteriophage exposed sample;
(b) determining a reference level of a bacteriophage nucleic acid;
(c) providing conditions to said combined microorganism and parent bacteriophage sufficient to allow said parent bacteriophage to infect said target microorganism to create an additional amount of said nucleic acid; and
(d) assaying a test level of said bacteriophage nucleic acid in said infected sample, and comparing said test level to said reference level to determine the presence or absence of said target microorganism in said sample.

27. A method as in claim 26 wherein said microorganism is a bacterium, and said assaying comprises detecting said nucleic acid as an indication of the presence of said target bacterium in said sample.

28. A method as in claim 26 wherein said assaying comprises a process selected from the group consisting of gel electrophoresis, oligo capture, fluorescence labeling, and calorimetric assays.

29. A method as in claim 26 wherein said assaying comprises amplifying said bacteriophage nucleic acid using an amplification method selected from the group consisting of PCR, SDA, or Rolling Circle.

30. A method as in claim 26 wherein said determining comprises:
providing a reference sample in which it is known that said target microorganism is not present;
adding said parent bacteriophage to said reference sample;
providing said conditions to said reference sample; and
assaying said reference sample to provide said reference level.

31. A method as in claim 26 wherein said determining comprises:
furnishing a reference portion of said sample;
killing said microorganism in said reference portion;
adding said parent bacteriophage to said reference sample;
providing said conditions to said reference sample; and
assaying said reference sample to provide said reference level.

32. A method as in claim 26 wherein said determining comprises a taking of a reference sub-sample from said bacteriophage exposed sample and performing a reference assay on said reference sub-sample before any bacteriophage amplification has occurred in said reference sub-sample.

33. A method of detecting the presence or absence of a target microorganism in a sample to be tested, said method comprising:
(a) combining with said sample, bacteriophage capable of infecting said target microorganism to create a bacteriophage exposed sample;
(b) providing conditions to said bacteriophage exposed sample sufficient to allow said bacteriophage to infect said target microorganism to create intermediate proteins associated with said replication of said bacteriophage, which intermediate proteins are not present in the fully replicated bacteriophage; and
(c) assaying said intermediate proteins to determine the presence or absence of said target microorganism.

34. A method as in claim 33 wherein said target microorganism is a bacterium, and said assaying comprises detecting said intermediate protein as an indication of the presence of said target bacterium in said sample.

35. A method as in claim 33 wherein said providing comprises dissociating said target microorganism prior to the completion of the replication cycle of said bacteriophage.

36. A method as in claim 33 wherein said sample is an insitu sample having an insitu matrix, said method further comprising isolating said microorganism from said insitu matrix prior to said assaying.

37. A method as in claim 36 wherein said isolating said microorganism is performed prior to said combining.

38. A method as in claim 37 wherein said isolating comprises capturing said microorganism and removing it from said matrix.

39. A method as in claim 38 wherein said capturing comprises phage mediated capturing.

40. A method of determining the presence or absence of a target microorganism in a sample to be tested, said method comprising:
(a) combining with said microorganism, an amount of parent bacteriophage capable of infecting said target microorganism to create a bacteriophage exposed sample;
(b) capturing said microorganism in said sample without using a bacteriophage or bacteriophage protein;
(c) providing conditions to said combined microorganism and parent bacteriophage sufficient to allow said bacteriophage to infect said target microorganism to create injected bacteriophage nucleic acid, additional bacteriophage nucleic acid, or additional bacteriophage protein; and
(d) assaying said injected bacteriophage nucleic acid, said additional bacteriophage nucleic acid or said additional bacteriophage protein to determine the presence or absence of said target microorganism in said insitu sample.

41. A method as in claim 40 wherein said microorganism is a bacterium, and said assaying comprises detecting said injected bacteriophage nucleic acid, said additional bacteriophage nucleic acid or said additional bacteriophage protein as an indication of the presence of said target bacterium in said sample.

42. A method as in claim 40 wherein said assaying comprises a process selected from the group consisting of gel electrophoresis, oligo capture, fluorescence labeling, and colorimetric assays.

43. A method as in claim 40 wherein said assaying is of said injected bacteriophage nucleic acid or said additional bacteriophage nucleic acid, and said assaying further comprises amplifying said injected bacteriophage nucleic acid or said additional bacteriophage nucleic acid using an amplification method selected from the group consisting of PCR, SDA, or Rolling Circle.

44. A method as in claim 40 and further including dissociating or lysing said microorganism prior to the completion of the replication cycle of said bacteriophage.

45. A method as in claim 40 wherein said capturing comprises immunomagnetic separation.

46. A method as in claim 40 wherein said capturing comprises utilizing an antibody to capture said microorganism.

47. A method as in claim 40 wherein said capturing comprises removing said microorganism from said sample.

48. A method as in claim 47 and further comprising cleaning said microorganism after said removing.

49. A method as in claim 48 and further comprising placing said cleaned microorganism in a sub-sample environment.

50. A method as in claim 49 wherein said combining includes placing said bacteriophage in a sub-sample environment.

51. A method as in claim 50 wherein said sub-sample environment is a liquid.

52. A method of detecting the presence or absence of a target microorganism in a test sample, said method comprising:
(a) combining with said microorganism, an amount of parent bacteriophage capable of infecting said target microorganism to create a bacteriophage exposed sample;
(b) providing conditions to said combined microorganism and parent bacteriophage sufficient to allow said parent bacteriophage to infect said target microorganism to create an additional amount of nucleic acid;
(c) assaying a test level of said bacteriophage nucleic acid in said infected sample, and comparing said test level to said reference level to determine the presence or absence of said target microorganism in said sample; and
(d) wherein said assaying comprises amplifying said bacteriophage nucleic acid using an amplification method selected from the group consisting of PCR, SDA, or Rolling Circle.

53. A method as in claim 52 wherein said assaying comprises a process selected from the group consisting of gel electrophoresis, oligo capture, fluorescence labeling, and colorimetric assays.

54. A method of detecting the presence or absence of a target microorganism in a sample to be tested, said method comprising:
(a) combining with said sample, bacteriophage that are not immobilized on a support, said bacteriophage capable of infecting said target microorganism to create a bacteriophage exposed sample;
(b) providing conditions to said bacteriophage exposed sample sufficient to allow said bacteriophage to infect said target microorganism to create injected bacteriophage nucleic acid or additional bacteriophage nucleic acid; and
(c) assaying said injected bacteriophage nucleic acid or said additional bacteriophage nucleic acid to determine the presence or absence of said target microorganism.
